(19) United States
(12) Patent Application Publication
(10) Pub. No.: US 2021/0235739 A1
(43) Pub. Date: Aug. 5, 2021

(54) NUTRITIONAL COMPOSITION FOR THE MANAGEMENT OF PHENYLKETONURIA AND METHOD OF PREPARATION

(71) Applicant: DR. SCHAR S.P.A., Postal (IT)

(72) Inventors: Virna Lucia Cerne, Postal (IT); Ombretta Polenghi, Postal (IT); Fabio Barban, Postal (IT)

(21) Appl. No.: 17/251,439

(22) PCT Filed: Jun. 13, 2019
(86) PCT No.: PCT/IT2019/050134

§ 371 Date: Dec. 11, 2020

(30) Foreign Application Priority Data
Jun. 13, 2018 (IT) 102018000006268

(51) Int. Cl. A23L 33/18; A23L 33/00; A23L 33/175; A23L 2/66; A23L 33/125; A23L 33/115

(52) U.S. Cl. CPC A23L 33/18; A23L 33/40; A23L 33/175; A23V 2002/00; A23L 33/125; A23L 33/115; A23L 2/66

(57) ABSTRACT

Nutritional formulation suitable to manage the diet for phenylketonuria, which comprises glycomacropeptide (GMP) as the primary source of proteins and additional complementary essential amino acids, to complete the required protein profile, supplied in a mixture comprising arginine, cystine, histidine, tryptophan, tyrosine and leucine.

### FIELD OF THE INVENTION

Embodiments described here concern a nutritional formulation to manage the diet for phenylketonuria, that is, to treat phenylketonuria, and the corresponding production method. The formulation according to the embodiments described here can be used for example to make a powder product to be dissolved in a liquid food, a beverage or suchlike, for example in water, before intake, that is, a powder to be reconstituted in the liquid. Furthermore, the formulation according to the embodiments described here can be used for example to also make a liquid product ready to be consumed, that is, ready to be drunk. Furthermore, the formulation according to the embodiments described here can for example be used as an ingredient to make other food products, such as dairy-like products, in particular yogurt, or confectionery products, such as creams or puddings, baked products, such as bread, crackers, breadsticks, snacks, biscuits, cakes, sweet snacks, patisserie and pasta products or suchlike, or other food drinks.

### BACKGROUND OF THE INVENTION

It is known that phenylketonuria (PKU) is a rare metabolic disease caused by a defect in phenylalanine metabolism. A deficiency in the phenylalanine hydroxylase enzyme or in its tetrahydrobiopterin cofactor causes an abnormal accumulation of phenylalanine in body fluids and in the central nervous system, which appears to be the part most affected by this pathology. Based on the residual capacity to metabolize phenylalanine and therefore the blood concentration of this amino acid, the disease is classified as severe, moderate or mild. If the blood concentration of phenylalanine is not controlled, patients experience cognitive deficit and irreversible mental retardation.

The average incidence of PKU in the Caucasian population is 1:10000/15000, with a peak in Turkey (1:2600) and a very low incidence in Finland (1:200000).

Following the diagnosis of PKU disease, a more or less restrictive protein free diet has to be started depending on the patient's residual enzyme activity and the protein intake has to be supplemented with foods for special medical purposes, free of phenylalanine. These foods are generally products in powder form to be reconstituted with water or liquid foods ready for use and consist of free amino acids, possibly carbohydrates and fats, mineral salts and trace elements and vitamins. The protein component normally consists of all the amino acids with the exception of phenylalanine and the amino acid profile is adapted according to the age group to which a specific product is addressed. It is essential that patients take these foods because they represent the only “safe” source of protein, that is, completely free of phenylalanine, containing a sufficient quantity of tyrosine and of other indispensable amino acids.

Foods for special medical purposes classically used in the treatment of PKU have several negative aspects:
organoleptic aspect: amino acids confer unpleasant off-flavors and off-odors to products, such as for example acidic, bitter, sulphureous or cabbage flavors or odors. Although patients become accustomed to these unusual tastes from infancy, there is a need to improve the organoleptic aspect of these products as well as the variety of products to choose from;
assimilation of the amino acids at the gastrointestinal level: unlike the absorption of normal proteins, the individual amino acids are absorbed much faster at the intestinal level since the protein hydrolysis step is not necessary. This results in a difference in nitrogen retention at body level and a reduced feeling of being full (correlated in many patients with the risk of being overweight).

Furthermore, the amino acids used to make these foods are obtained by hydrolysis of natural proteins or by means of chemical synthesis. The formulations therefore have a synthetic or artificial appearance in the eyes of consumers which manufacturers try to limit to the minimum necessary.

Since the 1990s, the possibility of using a particular peptide for the food treatment of PKU was discovered. The peptide, called glycomacropeptide (GMP), derives from the cutting of K-casein by chymosin during the production of cheese and can be easily isolated from whey. GMP is suitable for the food treatment of PKU, as it is naturally low in phenylalanine, therefore it can be integrated into food for special medical purposes as a source of protein.

The main positive aspects of GMP for the treatment of PKU are the following:
GMP is a peptide very similar to natural proteins and has a neutral taste, without the unpleasant tastes typical of amino acids. This characteristic allows the development of foods characterized by a better organoleptic profile than the amino acid mixtures traditionally used, which do not use GMP. Foods containing GMP help to increase the compliance of patients with the diet, thanks to the improvement in taste and the increase in products available for protein supplementation;
improvement in several aspects related to the assimilation of the peptide: the hydrolysis of the peptide ensures that the assimilation of amino acids occurs more slowly and physiologically, consequently increasing the sense of fullness of the patient. Furthermore, protein retention is increased and fluctuations in the levels of phenylalanine are reduced. This latter aspect consequently improves the blood control of phenylalanine with respect to a diet that only provides the intake of amino acids;
a significant level of LNAA (Long Neutral Amino Acids) compounds is found in GMP, a family of amino acids that share the same transporter as phenylalanine at the level of the blood-brain barrier. Leucine, valine, isoleucine, histidine, methionine, tryptophan, tyrosine and threonine belong to this family. The possibility of using mixtures of these amino acids that would saturate these transporters at brain level, limiting the entry of toxic phenylalanine into the brain, is known. Therefore, the use of LNAA mixtures to treat patients with PKU can decrease the concentration of phenylalanine accumulated at brain level, which is neurotoxic. Therefore, the LNAA present in GMP could have a protective role at brain level;
a diet comprising products with GMP promotes the growth of patients with PKU, favoring an increase in muscle mass and a reduction in fat mass.

It is also known that GMP is not completely free of phenylalanine and this limits the quantity of peptide that can be integrated in the products. Despite this limitation, the quantity of peptide that can be used in different products is sufficient to significantly improve their nutritional and organoleptic aspects, factors that positively influence the compliance of patients with the diet therapy. Furthermore, the current recommendation of dietitians is that GMP can be used to replace about 50% of protein intake in order to promote the nutritional value and palatability of the diet.

GMP is also low in other amino acids, such as arginine, cystine, histidine, tryptophan, tyrosine and leucine. Of these amino acids, histidine, tryptophan and leucine are essential, arginine, cystine and tyrosine are essential during the developmental phase of children. Tyrosine also becomes an essential amino acid throughout the entire life of people suffering from PKU, since it cannot be produced from phenylalanine as occurs under normal conditions. The amino acids lacking in GMP therefore have to be supplemented in order to meet the nutritional needs of patients with PKU.

Document WO-A-2010/144821 describes a medical food based on GMP for the nutritional management of PKU and other metabolic disorders, which, in addition to GMP, includes additional quantities of arginine, leucine and optionally other amino acids, such as tyrosine.

Document WO-A-2015/205489 describes a nutritional formulation in powder form that can be combined with water to make a milk-like beverage for the dietary management of PKU. The powder formulation comprises casein-GMP (cGMP) and complementary essential amino acids which are a mixture of tyrosine, arginine, tryptophan, leucine, histidine, which in combination give the required amino acid profile.

Document U.S. Pat. No. 9,414,619 describes a ready to drink liquid nutritional formulation to treat PKU, tyrosinemia or other metabolism errors. The formulation comprises cGMP, as a primary source of proteins, and complementary essential amino acids to complete the required protein profile. The beverage also includes vitamins and minerals to meet the daily needs of these elements and, when used as a complete diet, the formulation also includes a source of carbohydrates and a source of fats.

Document WO-A-98/08402 describes a nutritional formula for patients suffering from PKU which has, as a source of protein, a mixture of casein-GMP (cGMP) and complementary essential amino acids other than phenylalanine.

Document U.S. Pat. No. 6,355,612 describes a protein material with a low digestion rate, for the preparation of an enteral composition which makes it possible to modulate the levels of postprandial amino acids in the plasma.

Document FR-A-3.002.831 describes a food product ready for consumption for patients with a metabolic pathology of one or several amino acids.

The publication is also known “Position of the American Dietetic Association: Use of Nutritive and Nonnutritive Sweeteners”, Journal of the American Dietetic Association, Vol. 104, no. 2, 1 Feb. 2004, pages 255-275.

One disadvantage of known nutritional formulations is that, although they provide GMP and complementary essential amino acids for the purposes of diet therapy for PKU, they however have inadequate sensory properties, so that they are unpleasant from the organoleptic point of view for those who take the formulations described above.

There is therefore the need to perfect a nutritional formulation to manage the diet for phenylketonuria and the corresponding production method which can overcome at least one of the disadvantages of the state of the art.

In particular, one purpose of the present invention is to provide nutritional formulations which, in addition to providing GMP and complementary essential amino acids for the purpose of diet therapy for PKU, have adequate sensory properties, so as to be pleasant from the organoleptic point of view for those who take the formulations described above.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

In accordance with the above purposes, embodiments concern a nutritional formulation to manage the diet for phenylketonuria, in particular usable for the treatment of phenylketonuria. According to one embodiment, the nutritional formulation comprises glycomacropeptide (GMP) as the primary source of proteins and additional complementary essential amino acids, to complete the required protein profile, supplied in a mixture comprising arginine, cystine, histidine, tryptophan, tyrosine and leucine. Inside the nutritional formulation, the arginine is from 33 to 50 milligrams of arginine/grams of equivalent proteins and the leucine is from 47 to 76 milligrams of leucine/grams of equivalent proteins.

According to some embodiments, combinable with all the embodiments described here, inside the nutritional formulation, tyrosine is from 112 to 188 milligrams of tyrosine/grams of equivalent proteins.

According to some embodiments, combinable with all the embodiments described here, inside the nutritional formulation, the total weight of the additional complementary essential amino acids is from 35% to 65% of the total weight given by the weight of the GMP proteins together with the weight of the additional complementary essential amino acids.

According to some embodiments, combinable with all the embodiments described here, inside the nutritional formulation the tryptophan is from 12 to 22 milligrams of tryptophan/grams of equivalent proteins.

According to some embodiments, combinable with all the embodiments described here, inside the nutritional formulation histidine is from 22 to 37 milligrams of histidine/grams of equivalent proteins.

According to some embodiments, combinable with all the embodiments described here, inside the nutritional formulation the cystine is from 3.8 to 6.2 milligrams of cystine/grams of equivalent proteins.

According to some embodiments, combinable with all the embodiments described here, the arginine supplied as a complementary essential amino acid, in addition to the one deriving from GMP, derives from arginine supplied in the form of arginine salt, in particular arginine-aspartate salt.

Further embodiments described here concern a food product or beverage containing a formulation according to the present description.

According to some embodiments, combinable with all the embodiments described here, the food product or beverage also comprises carbohydrates and fats.

According to some embodiments, combinable with all the embodiments described here, carbohydrates provide from 20% to 50% of the energy of the product or beverage.

According to some embodiments, combinable with all the embodiments described here, fats provide from 1% to 50% of the energy of the product or beverage.

According to some embodiments, combinable with all the embodiments described here, the ratio of weight between proteins and carbohydrates in the product or beverage which includes the nutritional formulation ranges from 0.85 to 2.5.

According to some embodiments, combinable with all the embodiments described here, the ratio of weight between proteins and fats in the product or beverage which includes the nutritional formulation ranges from 10 to 130.

According to some embodiments, combinable with all the embodiments described here, the carbohydrates of the food product or beverage comprise trehalose, and trehalose is from 35% to 95% in weight with respect to the total quantity in weight of the carbohydrates present.

Other embodiments described here concern a method to make a nutritional formulation to manage the diet for phenylketonuria. According to one embodiment, the method comprises mixing glycomacropeptide (GMP) as a primary source of proteins, and additional complementary essential amino acids, to complete the required protein profile, supplied in a mixture comprising arginine, cystine, histidine, tryptophan, tyrosine and leucine. According to one embodiment, arginine and leucine are supplied in such quantities that, inside said nutritional formulation, arginine is from 33 to 50 milligrams of arginine/grams of equivalent proteins and leucine is from 47 to 76 milligrams of leucine/grams of equivalent proteins.

Other embodiments described here concern a method to make a food product or beverage to manage the diet for phenylketonuria. In some embodiments, this method provides to make available and mix together at least: a nutritional formulation in accordance with the present description, carbohydrates and fats. If a beverage is made, the method provides to mix the nutritional formulation, carbohydrates and fats made available with a food liquid, in particular water.

These and other aspects, characteristics and advantages of the present disclosure will be better understood with reference to the following description and attached claims.

The various aspects and characteristics described in the present description can be applied individually where possible. These individual aspects, for example aspects and characteristics in the description or in the attached dependent claims, can be the object of divisional applications.

It is understood that any aspect or characteristic that is discovered, during the patenting process, to be already known, shall not be claimed and shall be the object of a disclaimer.

### DETAILED DESCRIPTION OF SOME EMBODIMENTS

We will now refer in detail to the various embodiments of the present invention. Each example is supplied by way of illustration of the invention and shall not be understood as a limitation thereof. For example, the characteristics shown or described insomuch as they are part of one embodiment can be adopted on, or in association with, other embodiments to produce another embodiment. It is understood that the present invention shall include all such modifications and variants.

Before describing these embodiments, we must also clarify that the present description is not limited in its application to details of the construction and disposition of the components as described in the following description. The present description can provide other embodiments and can be obtained or executed in various other ways. We must also clarify that the phraseology and terminology used here is for the purposes of description only, and cannot be considered as limitative.

Unless otherwise defined, all the technical and scientific terms used here and hereafter have the same meaning as commonly understood by a person with ordinary experience in the field of the art to which the present invention belongs. Even if methods and materials similar or equivalent to those described here can be used in practice and in the trials of the present invention, the methods and materials are described hereafter as an example. In the event of conflict, the present application shall prevail, including its definitions. The materials, methods and examples have a purely illustrative purpose and shall not be understood restrictively.

In general, all the percentages and ratios indicated are intended to refer to the weight of the total composition (w/w), unless otherwise indicated.

All the percentage ranges shown here are provided with the provision that the sum with respect to the overall composition is at 100%, unless otherwise indicated.

All the ranges reported here shall be understood to include the extremes, including those that report a range “between” two values, unless otherwise indicated.

The present description also includes the ranges that derive from overlapping or uniting two or more ranges described, unless otherwise indicated.

The present description also includes the ranges that can derive from the combination of two or more values taken at different points, unless otherwise indicated.

Embodiments described here concern a nutritional formulation to manage the diet for phenylketonuria (PKU), having a nutritional profile of GMP protein and additional complementary amino acids to provide a balanced intake thereof for the diet for PKU.

In particular, the nutritional profile, that is, the composition, inside the nutritional formulation, of the essential amino acids can be expressed here in milligrams per grams of equivalent proteins (g EP or grams of equivalent proteins).

The total equivalent proteins (EP) can be calculated here as the sum of the GMP protein and additional complementary amino acids divided by 1.2, according to the formula:

EP=GMP protein+(sum of additional complementary amino acids/1.2).

The nutritional formulation described above comprises glycomacropeptide (GMP) as a primary source of proteins, and additional complementary essential amino acids, to complete the required protein profile. The additional complementary essential amino acids are supplied in a mixture comprising arginine, cystine, histidine, tryptophan, tyrosine and leucine.

Inside the nutritional formulation described above, arginine is from 33 to 50 milligrams of arginine/grams of equivalent proteins and leucine is from 47 to 76 milligrams of leucine/grams of equivalent proteins. Of these, the arginine that is added, in addition to that deriving from GMP, is from 32 to 48.5 milligrams of arginine/grams of equivalent proteins, while the leucine that is added, in addition to that deriving from GMP, is from 39 to 65 milligrams of leucine/grams of equivalent proteins.

The Applicant has found that leucine is an amino acid with an organoleptic profile with a very marked bitter taste. However, it is known that it plays an important nutritional role, being an essential amino acid. For these reasons, the dosage of leucine in the formulation according to the present description, overall from 47 to 76 milligrams of leucine/grams of equivalent proteins, is very reduced compared to the state of the art, with a minimum impact at the organoleptic level, in any case compensating the deficiency in the GMP, preserving the role of essential amino acid, as defined in the document “Protein and amino acids requirements in human nutrition” (WHO, Table 23 and Table 36).

The Applicant has found that arginine is also a very bitter amino acid. In the development step of the formulation according to the present description, the Applicant has therefore conducted experimental tests to compare the influence of different forms of arginine on the general organoleptic profile of the formulation with GMP in accordance with the present description. The Applicant has discovered that it is preferable to use the formulation of the arginine-aspartate salt compared to the pure form of arginine, since it is less bitter. Therefore, in some embodiments, the quantitative contribution of arginine that is added in the formulation described here, in addition to that deriving from GMP, derives from arginine supplied in the form of salt, in particular arginine-aspartate salt, helping to optimize the organoleptic profile of the product from the technological point of view

It is noted here that GMP intrinsically supplies reduced quantities of arginine, cystine, histidine and leucine, so that in the formulation described above, overall a minimal part of the arginine, cystine, histidine and leucine originate from GMP and the remaining larger part originate from additional complementary essential amino acids while, in the formulation described above, tryptophan and tyrosine originate only from the additional complementary essential amino acids.

According to possible embodiments, combinable with all the embodiments described here, inside the nutritional formulation, the tyrosine is from 112 to 188 milligrams of tyrosine/grams of equivalent proteins, originating exclusively from the additional amino acids and not from GMP.

According to possible embodiments, combinable with all the embodiments described here, inside the nutritional formulation, the total weight of the additional complementary essential amino acids is from 35% to 65% of the total weight given by the weight of the GMP proteins together with the weight of the additional complementary essential amino acids.

In further possible embodiments, the total weight of the additional essential complementary amino acids is from 45% to 65% of the total weight given by the weight of the GMP proteins together with the weight of the additional complementary essential amino acids.

In further possible embodiments, the total weight of the additional essential complementary amino acids is from 55% to 65% of the total weight given by the weight of the GMP proteins together with the weight of the additional complementary essential amino acids.

According to possible embodiments, combinable with all the embodiments described here, inside the nutritional formulation, tryptophan is from 12 to 22 milligrams of tryptophan/grams of equivalent proteins and histidine is from 22 to 37 milligrams of histidine/grams of equivalent proteins. Of these, tryptophan originates exclusively from the additional amino acids and not from GMP, while the histidine that is added, in addition to that deriving from GMP, is from 21 to 36.3 milligrams of histidine/grams of equivalent proteins.

According to possible embodiments, combinable with all the embodiments described here, inside the nutritional formulation, cystine is from 3.8 to 6.2 milligrams of cystine/grams of equivalent proteins. Of these, the cystine that is added, in addition to that deriving from GMP, is from 3.45 to 5.75 milligrams of cystine/grams of equivalent proteins.

In further embodiments, combinable with all the embodiments described here, inside the nutritional formulation described above, in addition to the amino acids mentioned above, arginine, cystine, histidine, leucine, tryptophan and tyrosine, one, more or all of the following amino acids are also present: alanine, aspartic acid, glutamic acid, glutamine, glycine, isoleucine, lysine, methionine, proline, serine, threonine, valine. Of these, isoleucine, serine and valine originate only from GMP, since intrinsically present therein, while alanine, aspartic acid, glutamic acid, glutamine, glycine, lysine, methionine, proline, threonine originate both from GMP, and also are added to complete the nutritional profile. Therefore, in possible embodiments, the nutritional formulation described above can also include, in addition to GMP and the amino acids intrinsically present therein, 15 additional amino acids, that is, arginine, cystine, histidine, leucine, tryptophan, tyrosine, in which GMP is low, and also alanine, aspartic acid, glutamic acid, glutamine, glycine, lysine, methionine, proline and threonine.

The minimal presence of phenylalanine in the formulation described here, in any case much below the minimum threshold for patients with PKU, originates exclusively from GMP where phenylalanine is intrinsically present. For example, GMP which contains a maximum of 0.2 mg of phenylalanine on 100 grams of protein is used. According to possible embodiments, combinable with all the embodiments described here, inside the nutritional formulation, phenylalanine is not greater than 1.6, preferably 1.5, even more preferably 1.4, milligrams of phenylalanine/grams of equivalent proteins. For example, phenylalanine can range from 1.2 to 1.5, preferably from 1.2 to 1.4, milligrams of phenylalanine/grams of equivalent proteins.

According to possible embodiments, combinable with all the embodiments described here, inside the nutritional formulation, alanine is from 51 to 75 milligrams of alanine/grams of equivalent proteins. Of these, the alanine that is added, in addition to that deriving from GMP, is from 28 to 47 milligrams of alanine/grams of equivalent proteins.

According to possible embodiments, combinable with all the embodiments described here, inside the nutritional formulation, aspartic acid is from 86 to 130 milligrams of aspartic acid/grams of equivalent proteins. Of these, the aspartic acid that is added, in addition to that deriving from GMP, is from 53 to 90 milligrams of aspartic acid/grams of equivalent proteins.

According to possible embodiments, combinable with all the embodiments described here, inside the nutritional formulation, glutamic acid is from 78 to 99.5 milligrams of glutamic acid/grams of equivalent proteins. Of these, the glutamic acid that is added, in addition to that deriving from GMP, is 7 to 12 milligrams of glutamic acid/grams of equivalent proteins.

According to possible embodiments, combinable with all the embodiments described here, inside the nutritional formulation, glutamine is from 47 to 79 milligrams of glutamine/grams of equivalent proteins, originating exclusively from the additional amino acids and not from GMP.

According to possible embodiments, combinable with all the embodiments described here, inside the nutritional formulation, glycine is from 58 to 96 milligrams of glycine/grams of equivalent proteins. Of these, the glycine that is added, in addition to that deriving from GMP, is from 54 to 91 milligrams of glycine/grams of equivalent proteins.

According to possible embodiments, combinable with all the embodiments described here, inside the nutritional formulation, isoleucine is from 39 to 96 milligrams of isoleucine/grams of equivalent proteins. Of these, the isoleucine that is added, in addition to that deriving from GMP, can be from 0 to 48 milligrams of isoleucine/grams of equivalent proteins.

According to possible embodiments, combinable with all the embodiments described here, inside the nutritional formulation, lysine is from 61 to 93 milligrams of lysine/grams of equivalent proteins. Of these, the lysine that is added, in addition to that deriving from GMP, is from 39 to 65 milligrams of lysine/grams of equivalent proteins.

According to possible embodiments, combinable with all the embodiments described here, inside the nutritional formulation, methionine is from 15 to 23.5 milligrams of methionine/grams of equivalent proteins. Of these, the methionine that is added, in addition to that deriving from GMP, is from 8 to 14 milligrams of methionine/grams of equivalent proteins.

According to possible embodiments, combinable with all the embodiments described here, inside the nutritional formulation, proline is from 99 to 146.5 milligrams of proline/grams of equivalent proteins. Of these, the proline that is added, in addition to that deriving from GMP, is from 55 to 92.5 milligrams of proline/grams of equivalent proteins.

According to possible embodiments, combinable with all the embodiments described here, inside the nutritional formulation, serine is from 28 to 69 milligrams of serine/grams of equivalent proteins. Of these, the serine that is added, in addition to that deriving from GMP, can be from 0 to 34.5 milligrams of serine/grams of equivalent proteins.

According to possible embodiments, combinable with all the embodiments described here, inside the nutritional formulation, threonine is from 79 to 105.5 milligrams of threonine/grams of equivalent proteins. Of these, the threonine that is added, in addition to that deriving from GMP, is from 16 to 28 milligrams of serine/grams of equivalent proteins.

According to possible embodiments, combinable with all the embodiments described here, inside the nutritional formulation, valine is from 31 to 77 milligrams of valine/grams of equivalent proteins. Of these, the valine that is added, in addition to that deriving from GMP, can be from 0 to 38.5 milligrams of valine/grams of equivalent proteins.

Advantageously, the aminogram that characterizes the formulations according to the various embodiments has been developed and tested by the Applicant in such a way that, as well as providing an adequate quantity of complementary essential amino acids suitable for the purposes of the diet therapy for PKU, at the same time it guarantees suitable sensory properties of the formulations themselves, so that they are also pleasing from the organoleptic point of view for those who take the formulations described above.

According to possible embodiments, combinable with all the embodiments described here, the nutritional formulation described above is in powder form. A possible example embodiment is a powder product containing the formulation according to the present description, to be taken once dissolved in a liquid, such as water or a similar suitable beverage. The powder product can include a mixture of dry ingredients, amongst which the formulation according to the present description and, for example, carbohydrates, fats, vitamins, mineral salts, trace elements or suchlike. The powder product can be incomplete or complete, depending on the recipe.

Further embodiments also concern a liquid product or food beverage containing a formulation in accordance with the embodiments described here. A possible example embodiment is a liquid product, or beverage, ready to drink, which contains at least the formulation in accordance with the embodiments and which can also contain, for example, vitamins and/or minerals and/or trace elements or other useful or functional substances. The liquid product can include a mixture of dry ingredients, including the formulation according to the present description, and of liquid ingredients. Among the additional ingredients, dry or liquid, in addition to the formulation according to the present description, for example, also in this case carbohydrates, fats, vitamins, mineral salts, trace elements or suchlike can be provided. The liquid product can be incomplete, or complete, depending on the recipe.

According to possible embodiments, other food products can also be provided which comprise the formulation according to the present description, such as dairy-like products, in particular yogurt, or confectionery products, such as creams or puddings, bakery products, such as bread, crackers, breadsticks, snacks, biscuits, cakes, sweet snacks, patisserie products and pasta or suchlike, or other formulations with different densities or viscosities. Other food beverages which use the nutritional formulation according to the present description can also be provided.

Therefore, providing that the formulation in accordance with the embodiments described here can be the “skeleton” or common base for the various food products or beverages that can be made comes within the spirit of the present description. This skeleton or common base can consist mainly of the protein part of GMP and additional complementary essential amino acids and therefore represents the heart of the various recipes for the different food products that can therefore be made.

Furthermore, the products obtainable which use, in the recipe, the formulation according to the embodiments described here, can have carbohydrates and fats added to them in varying quantities.

The products thus obtained can be complete products or incomplete products. If the product itself contains a quantity of proteins, carbohydrates and fats such as to supply on its own the nutritional needs of the patient with PKU, it is defined as complete, otherwise it is defined as incomplete.

According to possible embodiments, combinable with all the embodiments described here, in food products which include the nutritional formulation described above, carbohydrates and fats can also be present.

In these food products, carbohydrates can provide from 20% to 50% of the energy of the product, and fats can provide from 1% to 50% of the energy of the product.

With regards to carbohydrates, in possible embodiments, the use of monosaccharides is preferably limited as much as possible in the recipes for the food products obtainable, favoring the use of polysaccharides, such as for example starches, maltodextrin, trehalose and lactose.

In possible embodiments, in particular, the formulation can comprise trehalose from 35% to 95% in weight with respect to the total weight of the carbohydrates present in the food product which includes the nutritional formulation of the present description.

Trehalose, in particular, is a disaccharide consisting of two glucose molecules joined by an α-α (1,1)′ glycosidic bond. Its chemical structure makes it particularly stable from the kinetic and thermodynamic point of view and makes it a non-reducing sugar, therefore not very prone to react with other substances inside food matrices. In addition to this, trehalose has the ability to protect proteins and their structure in particular stress situations.

Furthermore, trehalose acts in the modulation of the salty and bitter taste in food matrices. Inside the formulation according to the present description, the use of trehalose has advantageously allowed to mask the bitter and salty tastes deriving not only from some amino acids but also from some mineral salts. It follows that the advantageous use of trehalose as the main source of carbohydrates also plays a role in the reduction of off-flavors typical of these formulations.

In the products obtainable according to the present description, the added carbohydrates can provide from about 20% to about 50% of the energy.

According to possible embodiments, combinable with all the embodiments described here, the ratio of weight between proteins and carbohydrates in the food product which includes the nutritional formulation of the present description can range from 0.85 to 2.5. Possible examples can be 0.85, 0.9, 0.95, 1, 1.25, 1.3, 1.5, 1.75, 2, 2.25, 2.5. These values, both in terms of range and also of specific values, can be adopted both for powder products and also for liquid products, according to needs.

According to possible embodiments, combinable with all the embodiments described here, the ratio of weight between proteins and fats in the food product which includes the nutritional formulation of the present description can range from 10 to 130. Possible examples can be 10, 12, 18, 25, 30, 35, 40, 45, 50, 60, 70, 80, 85, 90, 100, 110, 120, 130. In particular, in the case of a powder product, the ratio of weight between proteins and fats in the food product can range, for example, from 60 to 130. In the case of a liquid product, the ratio of weight between proteins and fats in the food product can range, for example, from 10 to 90.

With regards to fats, in the recipes for food products obtainable according to the present description, fats can be present, such as for example polyunsaturated essential fatty acids, in particular of the omega 3 class, for example alpha-linolenic acid, eicosapentaenoic acid (EPA), docosahexaenoic acid (DHA), and omega 6, for example linoleic acid and arachidonic acid (ARA), in order to enrich the nutritional profile of the products. For example, in recipes ARA, EPA and DHA can be used.

In addition, other vegetable fats, in particular vegetable oils, can also be provided in recipes to enrich the nutritional profile of the products. Possible examples are canola oil, sunflower oil, palm/palm kernel oil, soybean oil, linseed oil, safflower oil, coconut oil and shea oil.

Furthermore, the addition of intermediate chain fatty acids (Medium Chain Triglyceride, MCT) is also possible.

In the products obtainable according to the present description, the added fats can provide from about 1% to about 50% of the energy.

Furthermore, in the recipes for food products obtainable according to the present description, vegetable fibers can be present, up to about 10% in weight in the recipe.

The food products obtainable can also have minerals, trace elements and vitamins added to them, in order to obtain a balanced micronutrient profile.

Furthermore, other embodiments described here concern a method to make nutritional formulation to manage the diet for phenylketonuria. According to one embodiment, the method comprises mixing glycomacropeptide (GMP) as a primary source of proteins, and additional complementary essential amino acids, to complete the required protein profile, supplied in a mixture comprising arginine, cystine, histidine, tryptophan, tyrosine and leucine. Arginine and leucine are supplied in such quantities that, inside the nutritional formulation, arginine is from 33 to 50 milligrams of arginine/grams of equivalent proteins and leucine is from 47 to 76 milligrams of leucine/grams of equivalent proteins.

In further embodiments, a method can also be provided to produce food products, in powder or liquid form, using the formulation according to the present description.

If producing a dry powder product, the method can provide to mix the dry ingredients, including the formulation described above, in suitable mixers. Subsequently, the product is dosed and packaged in suitable containers, which can be multi-portion, such as tins or cans, or single-portion, preferably in modified atmosphere.

If producing a liquid product, ready to drink, the method can provide to mix in the selected liquid, for example water, the dry powdered ingredients, including the formulation described here, and the liquid ingredients. The liquid mixture obtained is mixed for a suitable time, after which it is microbiologically stabilized by means of thermal treatment (UHT or pasteurization) and packaged into packages of various volume, multi-portion or single-portion. Alternatively, the mixture can be stabilized by means of cold pasteurization with high pressure treatment (HHP, High Hydrostatic Pressure or HPP, High Pressure Processing); in this case, given the protein content, a variation in the density or viscosity of the final product can be obtained.

The aspects of the various products obtainable using the formulation and the method according to the present description, taken individually or in combination with each other according to the different embodiments described here, can therefore be:
very low phenylalanine content, not exceeding 1.4 milligrams of phenylalanine/grams of equivalent proteins;
protein part consisting exclusively of GMP and additional essential complementary amino acids;
aminogram in which, with respect to the total amino acids, from about 35% to about 65% derives from GMP and the remaining from about 65% to about 35% derives from the free amino acids. For example, about 40% derives from GMP and the remaining about 60% derives from free amino acids;
complete profile as regards all the amino acids, both essential and non-essential;
aminogram which allows to supply a quantity of complementary essential amino acids suitable for the purposes of the diet therapy for PKU, obtaining at the same time suitable sensory properties of the formulations, so that they are also pleasing from the organoleptic point of view for those who take the formulations described above, as described below with reference to the experimental tests performed by the Applicant;
high level of tyrosine, from 112 to 188 milligrams of tyrosine/grams of equivalent proteins, in line with the nutritional needs of patients with PKU, considering that, in PKU, tyrosine becomes an essential amino acid;
thanks to the peculiar amino acid composition, there is an adequate quantity of Long Neutral Amino Acids (LNAA) that can compete with the entrance of phenylalanine at the brain level. For example, in some embodiments, from about 0.360 to about 0.625 grams of LNAA/grams of equivalent proteins can be present. A possible example is about 0.448 grams of LNAA/grams of equivalent proteins.

The Applicant has found that, among the LNAAs, leucine is the one provided with the worst organoleptic profile, with a very marked bitter taste. However, the Applicant has also observed that it also plays an important nutritional role since it is an essential amino acid. For these reasons, the dosage of leucine in the formulation according to the present description, overall from 47 to 76 milligrams of leucine/grams of equivalent proteins, is considerably reduced compared to the state of the art, that is, it has a minimal impact at the organoleptic level and in any case compensates the deficiency in the GMP, preserving the role of essential amino acid.

Furthermore, the following peculiarities further characterize the products obtainable using the embodiments of the formulation described here:
in the case of incomplete products from the nutritional point of view, they can have a reduced caloric intake thanks to a reduced content of carbohydrates and above all of simple sugars;
the products can comprise a high content of micronutrients typically lacking in PKU patients (such as for example calcium, zinc, vitamin B12, selenium, iron), without exceeding in overdosing in order to respect the higher levels of intake established.
where possible, ingredients are added such as gluco-oligosaccharides/fructo-oligosaccharides GOS/FOS, prebiotic compounds such as inulin, and aronia melanocarpa (antioxidant) that, depending on the target patients, can improve the nutritional profile of the products in relation to PKU.

### EXAMPLES

### Example 1

The following Tables 1 and 2 show ranges of values of GMP and free amino acids present in formulations according to possible embodiments which can be suitable to produce various types of products, for example as described above. The values in Tables 1 and 2 are expressed as milligrams per gram of equivalent proteins.

Tables 1 and 2 considered as a whole provide examples in which the total weight of the additional complementary essential amino acids is from 45% to 65% of the total weight given by the weight of GMP proteins together with the weight of the additional complementary essential amino acids.

The GMP used contains 0.2 mg of phenylalanine on 100 grams of equivalent proteins and the upper limit of phenylalanine established is 1.4 mg/grams of equivalent proteins.

In particular, in Table 1 below, we refer to example embodiments in which the total weight of the additional essential complementary amino acids is from 55% to 65% of the total weight given by the weight of the GMP proteins together with the weight of the additional complementary essential amino acids.

TABLE 1

| | | Quantity of | Quantity of | |
|---|---|---|---|---|
| | Unit of | amino acid | free amino | Total |
| Amino acid | measure | from GMP | acids added | aminogram |
| Alanine | milligrams | 22.86-27.94 | 28.2-47 | 51.06-74.94 |
| | (mg) | | | |
| Arginine | milligrams | 1.08-1.32 | 32.85-48.18 | 33.93-49.5 |
| | (mg) | | | |
| Aspartic acid | milligrams | 32.58-39.82 | 53.85-89.75 | 86.43-129.57 |
| | (mg) | | | |
| Cystine | milligrams | 0.35-0.43 | 3.45-5.75 | 3.8-6.18 |
| | (mg) | | | |
| Glutamic acid | milligrams | 71.37-87.23 | 7.2-12 | 78.57-99.23 |
| | (mg) | | | |
| Glutamine | milligrams | — | 47.25-78.75 | 47.25-78.75 |
| | (mg) | | | |
| Glycine | milligrams | 4.14-5.06 | 54.3-90.5 | 58.44-95.56 |
| | (mg) | | | |
| Histidine | milligrams | 0.36-0.44 | 21.75-36.25 | 22.11-36.69 |
| | (mg) | | | |
| Isoleucine | milligrams | 39.15-47.85 | 0-47.85 | 39.15-95.7 |
| | (mg) | | | |
| Leucine | milligrams | 8.28-10.12 | 39-65 | 47.28-75.12 |
| | (mg) | | | |
| Lysine | milligrams | 22.86-27.94 | 39-65 | 61.86-92.94 |
| | (mg) | | | |
| Methionine | milligrams | 7.65-9.35 | 8.25-13.75 | 15.9-23.1 |
| | (mg) | | | |
| Proline | milligrams | 44.37-54.23 | 55.28-92.13 | 99.65-146.36 |
| | (mg) | | | |
| Serine | milligrams | 28.08-34.32 | 0-34.32 | 28.08-68.64 |
| | (mg) | | | |
| Threonine | milligrams | 63.45-77.55 | 16.5-27.5 | 79.95-105.05 |
| | (mg) | | | |
| Tryptophan | milligrams | — | 12.75-21.25 | 12.75-21.25 |
| | (mg) | | | |
| Tyrosine | milligrams | — | 112.5-187.5 | 112.5-187.5 |
| | (mg) | | | |
| Valine | milligrams | 31.5-38.5 | 0-38.5 | 31.5-77 |
| | (mg) | | | |
| Phenylalanine | milligrams | 1.26-1.54 | — | 1.26-1.54 |
| | (mg) | | | |

In Table 2 below, in particular, we refer instead to example embodiments in which the total weight of the additional complementary essential amino acids is from 45% to 55% of the total weight given by the weight of the GMP proteins together with the weight of the additional complementary essential amino acids.

TABLE 2

| | | Quantity of | Quantity of | |
|---|---|---|---|---|
| | Unit of | amino acid | free amino | Total |
| Amino acid | measure | from GMP | acids added | aminogram |
| Alanine | milligrams | 28.42-36.92 | 22.75-37.91 | 51.17-74.83 |
| | (mg) | | | |
| Arginine | milligrams | 1.29-1.68 | 32.64-47.87 | 33.93-49.54 |
| | (mg) | | | |
| Aspartic acid | milligrams | 40.48-52.58 | 46.10-76.84 | 86.58-129.42 |
| | (mg) | | | |
| Cystine | milligrams | 0.43-0.56 | 3.38-5.63 | 3.81-6.19 |
| | (mg) | | | |
| Glutamic acid | milligrams | 88.71-115.23 | 10.09-16.82 | 98.81-132.05 |
| | (mg) | | | |
| Glutamine | milligrams | — | 47.29-78.82 | 47.29-78.82 |
| | (mg) | | | |
| Glycine | milligrams | 5.17-6.71 | 53.30-88.83 | 58.46-95.54 |
| | (mg) | | | |
| Histidine | milligrams | 0.43-0.56 | 22.23-35.63 | 22.66-36.19 |
| | (mg) | | | |
| Isoleucine | milligrams | 48.66-6321 | 0-63.20 | 48.66-126.41 |
| | (mg) | | | |
| Leucine | milligrams | 10.34-13.42 | 36.84-61.4 | 47.18-74.82 |
| | (mg) | | | |
| Lysine | milligrams | 28.42-36.92 | 33.25-55.41 | 61.67-92.33 |
| | (mg) | | | |
| Methionine | milligrams | 9.47-12.31 | 6.08-10.14 | 15.56-22.44 |
| | (mg) | | | |
| Proline | milligrams | 55.12-71.60 | 44.73-74.55 | 99.85-146.15 |
| | (mg) | | | |
| Serine | milligrams | 34.88-45.31 | 0-45.31 | 34.88-90.61 |
| | (mg) | | | |
| Threonine | milligrams | 78.81-102.36 | 1.06-1.77 | 79.87-104.13 |
| | (mg) | | | |
| Tryptophan | milligrams | — | 12.75-21.25 | 12.75-21.25 |
| | (mg) | | | |
| Tyrosine | milligrams | — | 112.5-187.5 | 112.5-187.5 |
| | (mg) | | | |
| Valine | milligrams | 39.19-50.90 | 0-50.90 | 39.19-101.80 |
| | (mg) | | | |
| Phenylalanine | milligrams | 1.57-2.03 | — | 1.57-2.03 |
| | (mg) | | | |

The following Table 3, instead, shows the values of GMP and free amino acids present in a specific formulation example according to the present description. The values in Table 3 are expressed as milligrams per gram of equivalent proteins.

TABLE 3

| | | Quantity of | Quantity of | |
|---|---|---|---|---|
| | Unit of | amino acid | free amino | Total |
| Amino acid | measure | from GMP | acids added | aminogram |
| Alanine | milligrams | 25.4 | 37.6 | 63 |
| | (mg) | | | |
| Arginine | milligrams | 1.2 | 43.8 | 45 |
| | (mg) | | | |
| Aspartic acid | milligrams | 36.2 | 71.8 | 108 |
| | (mg) | | | |
| Cystine | milligrams | 0.39 | 4.6 | 5 |
| | (mg) | | | |
| Glutamic acid | milligrams | 79.3 | 9.6 | 89 |
| | (mg) | | | |
| Glutamine | milligrams | — | 63 | 63 |
| | (mg) | | | |
| Glycine | milligrams | 4.6 | 72.4 | 77 |
| | (mg) | | | |
| Histidine | milligrams | 0.4 | 29 | 29 |
| | (mg) | | | |
| Isoleucine | milligrams | 43.5 | — | 44 |
| | (mg) | | | |
| Leucine | milligrams | 9.2 | 52 | 61 |
| | (mg) | | | |
| Lysine | milligrams | 25.4 | 52 | 77 |
| | (mg) | | | |
| Methionine | milligrams | 8.5 | 11 | 19 |
| | (mg) | | | |
| Proline | milligrams | 49.3 | 73.7 | 123 |
| | (mg) | | | |
| Serine | milligrams | 31.2 | — | 31 |
| | (mg) | | | |
| Threonine | milligrams | 70.5 | 22 | 92 |
| | (mg) | | | |
| Tryptophan | milligrams | — | 17 | 17 |
| | (mg) | | | |
| Tyrosine | milligrams | — | 150 | 150 |
| | (mg) | | | |
| Valine | milligrams | 35 | — | 35 |
| | (mg) | | | |
| Phenylalanine | milligrams | 1.4 | — | 1.4 |
| | (mg) | | | |

### Example 2

Table 4 below shows an example of a powdered food product comprising a nutritional formulation according to the present description and containing GMP, to be consumed after reconstitution with water. This example is an incomplete product which provides about 300 kcal/100 g and is characterized by the following macronutrient profile, expressed as a range of quantities in grams per 100 g of powdered food product.

TABLE 4

| | g/100 g |
|---|---|
| Proteins | 30-45 |
| Carbohydrates | 20-35 |
| Fats | 0.35-2 |

This example of a powdered food product according to the present description can consist of the following ingredients, expressed as a quantity in grams per 100 g of powdered food product (Table 5).

TABLE 5

| | g/100 g |
|---|---|
| Alanine | 1.6 |
| Cystine | 0.2 |
| Glutamine | 2.8 |
| Glycine | 3.2 |
| Histidine | 1.3 |
| Leucine | 2.3 |
| Methionine | 0.5 |
| Proline | 3.3 |
| Threonine | 1 |
| Tryptophan | 0.8 |
| Tyrosine | 6.6 |
| Arginine aspartate | 3.4 |
| Lysine aspartate | 3.6 |
| Lysine glutamate | 1 |
| GMP | 22 |
| Trehalose | 20 |
| GOS | 1.7 |
| FOS | 0.2 |
| DHA | 2.6 |
| Potassium carbonate | 1.5 |
| Calcium phosphate | 4.6 |
| Potassium phosphate | 1.7 |
| Magnesium carbonate | 1.2 |
| Iron sulfate | 1.6 |
| Zinc sulfate | 0.04 |
| Copper sulfate | 0.003 |
| Manganese sulfate | 0.005 |
| Sodium selenite | 0.0002 |
| Chromium chloride | 0.0003 |
| Sodium molybdate | 0.0002 |
| Potassium iodide | 0.0004 |
| Carnitine | 0.04 |
| Choline | 1 |
| Inositol | 0.13 |
| Vitamins | 0.7 |
| Sucralose | 0.04 |
| Aromas | 12 |

### Example 3

An example of a liquid food product comprising a nutritional formulation according to the present description and containing GMP, ready for consumption, is shown below in Table 6. This example is an incomplete product which provides about 60 kcal/100 g and is characterized by the following macronutrient profile, expressed as a range of quantities in grams per 100 g of liquid food product.

TABLE 6

| | g/100 g |
|---|---|
| Proteins | 6-9 |
| Carbohydrates | 4-6 |
| Fats | 0.1-1 |

This example of a liquid product according to the present description can consist of the following ingredients, expressed as a quantity in grams per 100 g of powdered food product (Table 7).

TABLE 7

| | g/100 g |
|---|---|
| Alanine | 0.3 |
| Cystine | 0.035 |
| Glutamine | 0.5 |
| Glycine | 0.55 |
| Histidine | 0.2 |
| Leucine | 0.4 |
| Methionine | 0.08 |
| Proline | 0.6 |
| Threonine | 0.15 |
| Tryptophan | 0.13 |
| Tyrosine | 1.13 |
| Arginine aspartate | 0.6 |
| Lysine aspartate | 0.61 |
| Lysine glutamate | 0.15 |
| GMP | 3.8 |
| Trehalose | 2.8 |
| Starch | 2.1 |
| Inulin | 2 |
| DHA | 0.15 |
| Sodium citrate | 0.4 |
| Potassium phosphate | 0.23 |
| Calcium phosphate | 0.7 |
| Magnesium carbonate | 0.2 |
| Potassium chloride | 0.3 |
| Iron sulfate | 0.025 |
| Zinc sulfate | 0.006 |
| Copper sulfate | 0.0006 |
| Manganese sulfate | 0.0007 |
| Sodium selenite | 0.00004 |
| Chromium chloride | 0.00004 |
| Sodium molybdate | 0.00003 |
| Potassium iodide | 0.00006 |
| Carnitine | 0.006 |
| Choline | 0.2 |
| Inositol | 0.02 |
| Vitamins | 0.1 |
| Aromas | 0.7 |
| Dye | 0.01 |
| Aronia melanocarpa | 0.004 |
| Sucralose | 0.01 |
| Sunflower lecithin | 0.4 |
| Water | 80.4 |

### EXPERIMENTAL TESTS

The formulation according to the present description is complex and provides the presence of different ingredients such as GMP, free amino acids, as well as possible carbohydrates, minerals and trace elements. It follows that the final taste of the formulation depends on the taste of the individual ingredients used, on their interaction in the matrix and possibly on the technological processes that the food undergoes during production. The formulation according to the present description has been developed and experimentally studied by the Applicant in order to optimize each of these aspects and make the products as stable as possible throughout their entire shelf life.

As evidence of these organoleptic improvements, the Applicant carried out a sensory evaluation of the powder formulation according to the examples of Tables 3 and 4, with respect to two other products with GMP (Product 1 and Product 2).

The following Table 8 summarizes the characteristics of the example powder formulation according to the present description, and of the Products 1 and 2 compared.

TABLE 8

| | Formulation according | | |
|---|---|---|---|
| | to an example of the | Product | Product |
| Product characteristics | present description | 1 | 2 |
| Energy kcal/10 g EP | 75 kcal | 60 kcal | 106 kcal |
| Energy from proteins | 54% | 66% | 38% |
| (%) | | | |
| Energy from | 40% | 21% | 57% |
| carbohydrates (%) | | | |
| Energy from fats (%) | 5% | 13% | 25% |
| g proteins/100 g | 43 g | 56 g | 31 g |
| g carbohydrates/100 g | 32 g | 18 g | 47 g |
| g fats/100 g | 1.7 g | 4.7 g | 9.2 g |

The sensory analysis was carried out by a trained panel consisting of 9 tasters. The parameters were evaluated using the following scale:

| Parameter intensity | Significance |
|---|---|
| 0 | Not perceptible |
| 1 | Slightly perceptible |
| 2 | Moderately perceptible |
| 3 | Clearly perceptible |
| 4 | Very perceptible |
| 5 | Extremely perceptible |

As shown in Table 9 below, the tasting of the sensory analysis carried out showed that the intensity of the bitter and acid tastes is much lower in the powder formulation according to the example of the present invention than in the two Products 1 and 2 compared.

TABLE 9

| | Formulation according | | |
|---|---|---|---|
| | to an example of the | Product | Product |
| | present description | 1 | 2 |
| Bitter | 1.1 | 2.3 | 3.2 |
| taste | | | |
| Acid | 1.3 | 1.7 | 1.9 |
| taste | | | |

It is clear that modifications and/or additions of parts and/or steps may be made to the nutritional formulation to manage the diet for phenylketonuria and corresponding production method as described heretofore, without departing from the field and scope of the present invention.

It is also clear that, although the present invention has been described with reference to some specific examples, a person of skill in the art shall certainly be able to achieve many other equivalent forms of nutritional formulation to manage the diet for phenylketonuria and corresponding production method, having the characteristics as set forth in the claims and hence all coming within the field of protection defined thereby.

1. A nutritional formulation suitable to manage the diet for phenylketonuria, said nutritional formulation comprising: glycomacropeptide (GMP) as the primary source of proteins and additional complementary essential amino acids, to complete the required protein profile, supplied in a mixture comprising arginine, cystine, histidine, tryptophan, tyrosine and leucine, in which, inside said nutritional formulation, the arginine is from 33 to 50 milligrams of arginine/grams of equivalent proteins and the leucine is from 47 to 76 milligrams of leucine/grams of equivalent proteins.

2. The nutritional formulation of claim 1, wherein, inside said nutritional formulation, the tyrosine is from 112 to 188 milligrams of tyrosine/grams of equivalent proteins.

3. The nutritional formulation of claim 1, wherein, inside said nutritional formulation, the total weight of said additional complementary essential amino acids is from 35% to 65% of the total weight given by the weight of the GMP proteins together with the weight of said additional complementary essential amino acids.

4. The nutritional formulation of claim 1, wherein, inside said nutritional formulation, the tryptophan is from 12 to 22 milligrams of tryptophan/grams of equivalent proteins, the histidine is from 22 to 37 milligrams of histidine/grams of equivalent proteins and the cystine is from 3.8 to 6.2 milligrams of cystine/grams of equivalent proteins.

5. The nutritional formulation of claim 1, wherein said arginine, supplied as a complementary essential amino acid, as well as the one deriving from GMP, derives from arginine supplied in form of arginine salt, in particular arginine-aspartate salt.

6. A food product or beverage containing a formulation according to claim 1.

7. A food product or beverage as in claim 6, also comprising carbohydrates and fats, where the carbohydrates supply from 20% to 50% of the energy of said product or beverage and the fats supply from 1% to 50% of the energy of said product or beverage.

8. A food product or beverage as in claim 6, wherein the ratio of weight between proteins and carbohydrates in said product or beverage that includes said nutritional formulation ranges from 0.85 to 2.5 and the ratio of weight between proteins and fats in said product or beverage that includes said nutritional formulation ranges from 10 to 130.

9. A food product or beverage as in claim 6, wherein the carbohydrates comprise trehalose and the trehalose is from 35% to 95% in weight with respect to the quantity of overall weight of the carbohydrates present in said product or beverage.

10. A method of making a nutritional formulation suitable to manage the diet for phenylketonuria, said method comprising: mixing glycomacropeptide (GMP) as the primary source of proteins, and additional complementary essential amino acids, to complete the required protein profile, supplied in a mixture comprising arginine, cystine, histidine, tryptophan, tyrosine and leucine, in which arginine and leucine are supplied in such quantities that, inside said nutritional formulation, the arginine is from 33 to 50 milligrams of arginine/grams of equivalent proteins and the leucine is from 47 to 76 milligrams of leucine/grams of equivalent proteins.

11. A method of making a food product or beverage suitable to manage the diet for phenylketonuria, said method providing to make available and mix together at least: a nutritional formulation according to claim 1, carbohydrates and fats.
